(19) United States
(12) Patent Application Publication
(10) Pub. No.: US 2024/0105338 A1
(43) Pub. Date: Mar. 28, 2024

(54) PREDICTING STATES FROM PATIENT-CAREGIVER DYADIC BIOMARKER DATA USING ARTIFICIAL INTELLIGENCE

(71) Applicant: International Business Machines Corporation, Armonk, NY (US)

(72) Inventors: William Ogallo, Nairobi (KE); Julian Bertram Kuehnert, Nairobi (KE); Sekou Lionel Remy, Nairobi (KE); Isaiah Mulang' Onando, Nairobi (KE)

(21) Appl. No.: 17/950,464

(22) Filed: Sep. 22, 2022

(51) Int. Cl. G16H 50/30; G06N 7/00

(52) U.S. Cl. CPC G16H 50/30; G06N 7/005

(57) ABSTRACT

Methods, systems, and computer program products for predicting states from patient-caregiver dyadic biomarker data using artificial intelligence are provided herein. A computer-implemented method includes obtaining biomarker data derived from one or more dyads, each dyad comprising at least one patient and at least one caregiver associated with the at least one patient; determining, based on processing the obtained biomarker data, data-based representations of mental distress and/or social rhythm disruption among at least one of the one or more dyads; predicting mental distress and/or social rhythm disruption among a given dyad of at least one patient and at least one caregiver associated with the at least one patient by processing input biomarker data, derived from the given dyad, using artificial intelligence techniques in connection with at least a portion of the data-based representations; and performing automated actions based on the predicting.

### BACKGROUND

The present application generally relates to information technology and, more particularly, to data processing techniques. More specifically, illness and/or injury which results in separation and/or social isolation of patients can have significant psychological consequences for patients and caregivers. For example, such separation can lead to various disorders such as, for example, anxiety, depression, insomnia, etc.

However, conventional mental health assessment and management procedures are resource-intensive, time-intensive, and error-prone. For example, such conventional procedures typically focus exclusively on a limited set of data pertaining to the patient(s) in question, and lack the ability to process and integrate caregiver-related data.

### SUMMARY

In at least one embodiment, techniques for predicting states from patient-caregiver dyadic biomarker data using artificial intelligence are provided. An example computer-implemented method includes obtaining biomarker data derived from a set of one or more dyads, each dyad comprising at least one patient and at least one caregiver associated with the at least one patient, and determining, based at least in part on processing at least a portion of the obtained biomarker data, one or more data-based representations of at least one of mental distress among at least one of the one or more dyads and social rhythm disruption among at least one of the one or more dyads. The method also includes predicting at least one of mental distress among a given dyad of at least one patient and at least one caregiver associated with the at least one patient and social rhythm disruption among the given dyad by processing input biomarker data, derived from the given dyad, using one or more artificial intelligence techniques in connection with at least a portion of the one or more data-based representations. Further, the method includes performing one or more automated actions based at least in part on the predicting step.

In one or more embodiments, determining one or more data-based representations includes performing phenotypic characterization by processing at least a portion of the obtained biomarker data using one or more artificial intelligence-based representation learning techniques. Also, in at least one embodiment, predicting mental distress and/or social rhythm disruption among a given dyad includes processing input biomarker data using one or more multivariate time series modeling techniques with one or more probabilistic transformers, in connection with one or more data-based representations. Further, in one or more embodiments, performing one or more automated actions includes automatically initiating, based at least in part on predicting mental distress and/or social rhythm disruption among a given dyad, one or more avatar-mediated interactions, one or more robot-mediated interactions, and/or one or more human-mediated interactions within at least a portion of the given dyad.

Another embodiment of the invention or elements thereof can be implemented in the form of a computer program product tangibly embodying computer readable instructions which, when implemented, cause a computer to carry out a plurality of method steps, as described herein. Furthermore, another embodiment of the invention or elements thereof can be implemented in the form of a system including a memory and at least one processor that is coupled to the memory and configured to perform noted method steps. Yet further, another embodiment of the invention or elements thereof can be implemented in the form of means for carrying out the method steps described herein, or elements thereof; the means can include hardware module(s) or a combination of hardware and software modules, wherein the software modules are stored in a tangible computer-readable storage medium (or multiple such media).

Illustrative embodiments can provide significant advantages relative to conventional mental health assessment and management procedures. For example, problems associated with resource-intensive, time-intensive, and error-prone techniques are overcome in one or more embodiments through dynamically predicting states of mental distress and social rhythm disruptions by processing patient-caregiver dyadic biomarker data using artificial intelligence techniques.

These and other objects, features and advantages of the present invention will become apparent from the following detailed description of illustrative embodiments thereof, which is to be read in connection with the accompanying drawings.

### BRIEF DESCRIPTION OF THE DRAWINGS

FIG. 1 is a block diagram according to an example embodiment of the invention;

FIG. 2 is a diagram illustrating an illustrative model of confirmatory factor analysis of associations between observed biomarkers and health-related factors, according to an example embodiment of the invention;

FIG. 3 is a flow diagram illustrating techniques according to an embodiment of the present invention;

FIG. 4 is a flow diagram illustrating techniques according to an example embodiment of the invention; and

FIG. 5 is a diagram illustrating a computing environment in which at least one embodiment of the invention can be implemented.

### DETAILED DESCRIPTION

At least one embodiment includes techniques related to dyadic phenotyping, prognostication, and supportive care of mental distress in contexts of clinically affective isolation. As used herein, dyadic phenotyping refers to a process of extracting and/or deriving composites, features, characteristics, and/or biomarkers from clinical and/or other health data of pairs of individuals that are linked (e.g., linked by a family relationship, lined by a patient-caregiver relationship, etc.). More specifically, one or more embodiments include implementing artificial intelligence-based methods of analyzing objectively measured dyadic sequence data to enable detection and/or amelioration of mental distress in one or more patients and/or one or more caregivers. Additionally, such an embodiment can include representing states of mental distress and/or social rhythm disruptions based at least in part on processing patient-caregiver dyadic biomarker data using one or more artificial intelligence techniques. In such a context as detailed herein, social rhythm refers to a level of engagement by a person in connection with social activities and/or habitual behaviors.

As further detailed herein, one or more embodiments include processing a plurality of clinical and/or digital biomarker data of patients and caregivers, such biomarkers including, for example, one or more physiologic measurements, imaging data, audio data, gesture-related data, movement-related data, sleep pattern data, etc. Based at least in part on the processing of such biomarker data, at least one embodiment includes performing phenotypic characterization of one or more of the given biomarkers as representative of mental distress, given patient-caregiver dyad information. Examples of patient-caregiver dyad information include, but are not limited to, anxiety-related information, self-reported stress information, physical health characteristics (e.g., blood pressure), etc. Such a characterization can be performed and/or implemented using data-driven approaches such as, for example, classification via one or more deep neural networks, fuzzy and/or deterministic rule-based models, etc.

Additionally, one or more embodiments include analyzing dyadic phenotype data to predict levels of mental distress and/or social rhythm in patients and/or caregivers, wherein one or more predictive models can be implemented which are informed by data-driven and/or expert-defined parameters.

Further, such an embodiment can include automatically initiating and/or triggering digital and/or avatar-mediated distanced interactions between one or more isolated patients and one or more caregivers based at least in part on the characterized and/or predicted levels of mental distress and one or more social interaction constraints.

Accordingly, at least one embodiment includes assessing patient and caregiver needs for targeted social interaction(s) to prevent or reduce mental distress. Such an embodiment includes analyzing data pertaining to the mental state of the patient(s) and the caregiver(s), as well as information related to the patient-caregiver relationship, in order to implement and manage one or more beneficial interactions. As used herein, information on patient-caregiver relationships can include, but is not limited to, relationship type (e.g., siblings, parent-child, friend, professional, etc.), duration, quality of the relationship as captured in the system, etc. Such analysis can further include, for example, consideration of the timing and duration of one or more candidate interactions, as well as the type of interaction for which different media are available. Additionally or alternatively, one or more embodiments can include initiating and/or triggering urgent intervention(s) based at least in part on individually assigned and/or individually learned signal thresholds related to the start or the stop of an interaction (and which may also be related to activating audio and/or video playback or other sensory stimulation).

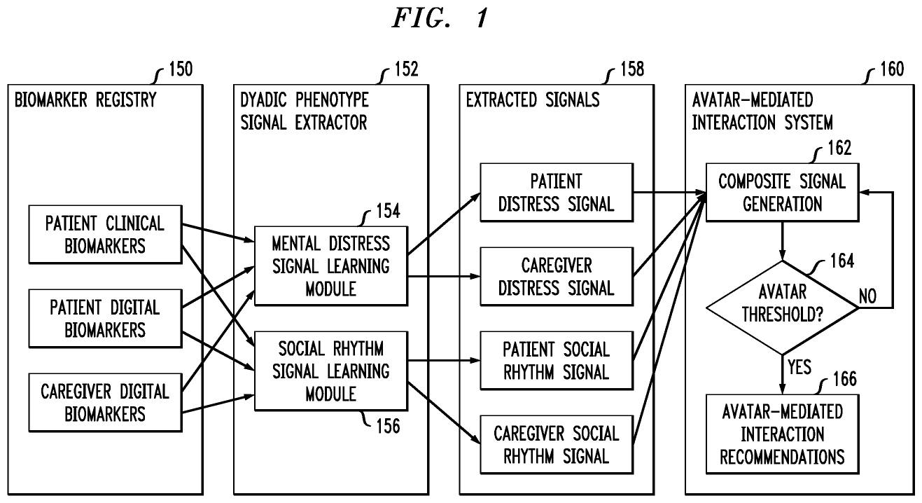

FIG. 1 is a block diagram according to an example embodiment of the invention. By way of illustration, FIG. 1 depicts a biomarker registry 150 that maintains a plurality of biomarker data captured via different modalities, including, for example, patient clinical biomarkers, patient digital biomarkers, and caregiver digital biomarkers. From this registry, one or more candidate biomarkers of mental distress can be identified that indicate a prognosis of patient-caregiver dyads.

Additionally, such dyadic phenotypes are analyzed to predict one or more levels related to mental distress and social rhythm information (e.g., measures of the regularity of patient and/or caregiver engagement in social activities and/or social interaction over time) among patients and caregivers using one or more predictive models. Specifically, and as further detailed herein, FIG. 1 depicts biomarker data from biomarker registry 150 being processed by learning modules within dyadic phenotype signal extractor 152. As illustrated in FIG. 1, such modules can include mental distress signal learning module 154 and social rhythm signal learning module 156, and such modules can determine and/or generate extracted signals 158 which can include, for example, patient distress signals, caregiver distress signals, patient social rhythm signals, and caregiver social rhythm signals.

Using at least a portion of the extracted signals 158, an avatar-mediated interaction system 160 generates a composite signal via step 162 that can be used in one or more ways. Such a composite signal can be generated by way of one or more representation learning methods that extract one or more latent biomarker representations of mental distress and social rhythm disruption among patient-caregiver dyads. For example, and as depicted in the FIG. 1 example, using the generated composite signal, avatar-mediated interaction system 160 can determine, via step 164, whether an avatar threshold (e.g., a signal associated with the composite signal) has been surpassed. If yes, then step 166 includes generating one or more avatar-mediated interaction recommendations for the patient and/or caregiver. If no, then avatar-mediated interaction system 160 reverts to step 162 (e.g., and awaits additional signal data). The composite signal can also be used, by way of example, to determine one or more constraints to interventions used to mitigate poor prognoses of mental distress and/or enhance prognoses given the patient-caregiver dyadic context.

Implementation of one or more embodiments can include, for example, utilization of one or more patient-side infrastructure and/or devices and one or more caregiver-side infrastructure and/or devices. For instance, patient-side infrastructure and/or devices can include devices which allow the detection of biomarkers, including cameras, microphones, wearables, sensory mattresses, etc. For direct interactions with one or more caregivers, for example, cameras and microphones can be used. Capturing data related to indirect interactions with one or more caregivers can be enabled, for example, using recording and interpretation of mimicries and/or gestures, which can then be translated to one or more avatar actions. Examples of such mimicries and/or gestures include, but are not limited to, imitations such as body postures, hand gestures, and/or other motor movements.

Additionally, in one or more embodiments, caregiver-side infrastructure and/or devices can include, for instance, mobile wearables, smartphones, etc. Interaction requests can be captured, for example, via smartphone notifications, while interaction data can be captured using smartphone cameras and/or microphones.

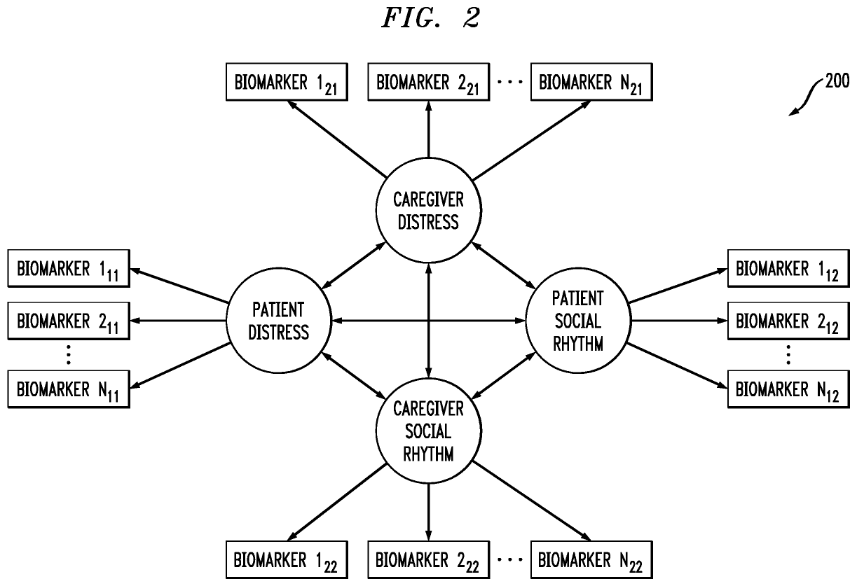

FIG. 2 is a diagram illustrating an illustrative model of confirmatory factor analysis of associations between observed biomarkers and health-related factors, according to an example embodiment of the invention. More specifically, FIG. 2 depicts a conceptual model 200 of confirmatory factor analysis of associations between observed biomarkers and latent factors related to patient distress, patient social rhythm, caregiver distress, and caregiver social rhythm. In one or more embodiments, model 200 can be used to verify how well observable and measurable features (biomarkers) obtained from patient-caregiver dyads represent the hidden constructs pertaining to the dyad's mental distress and social rhythm.

Such biomarker data as detailed in connection with FIG. 2 can, in one or more embodiments, be maintained in at least one biomarker registry (e.g., biomarker registry 150 as depicted in FIG. 1). Such a biomarker registry comprises and/or maintains a plurality of measurable clinical and/or digital biomarkers from multiple patient-caregiver dyads. For example, patient clinical biomarkers can include, but are not limited to, physiological biomarkers such as blood pressure, heart rate, temperature, and brain-related measurements (e.g., electroencephalographic activity), inflammatory biomarkers such as heat shock proteins, acute phase proteins, and C-reactive protein, as well as protein biomarkers such as cortisol and adrenaline. Additionally, patient digital biomarkers can include, but are not limited to, mobility data, physical activity data, sleep pattern data, body sweat diagnostic data, voice feature data, and speech feature data. Similarly, caregiver digital biomarkers can include, but are not limited to, mobility data, physical activity data, sleep pattern data, body sweat diagnostic data, voice feature data, and speech feature data.

Referring again to FIG. 1, in at least one embodiment, dyadic phenotype signal extractor 152 identifies, using at least a portion of the data maintained in biomarker registry 150, one or more relevant clinical and/or digital biomarkers that are associated with mental distress and/or social rhythm disruption of one or more patients and/or one or more caregivers. In such an embodiment, this can be achieved by way of exploratory and confirmatory factor analysis of the association between prognostic biomarkers and latent factors (such as illustrated, for example, in FIG. 2). For example, longitudinal data (e.g., scores and/or scales) on latent factors such as patient mental distress, patient social rhythm disruption, caregiver mental distress, and caregiver social rhythm disruption can be derived from questionnaires developed by domain experts and administered (e.g., periodically) to patient and caregiver dyads.

As further detailed herein, one or more embodiments include using representation learning techniques to extract mental distress signals and social rhythm disruption signals representations among patient-caregiver dyads. An example of such a representation learning technique can include supervised multimodal deep representational learning techniques using one or more artificial neural networks and multimodal learning with one or more transformers. In such an embodiment, at least one probabilistic transformer can be implemented to model mental distress and social rhythm disruption as an underlying latent dyadic process that generates observed changes over time. By way of example, a probabilistic transformer can include a supervised learning algorithm that combines deep learning with state space modeling. The inputs to such a model can include multivariate time series feature vectors derived from representations of mental distress and social rhythm disruption. Additionally, the model uses an encoder to project the input vectors to a latent space and additional transformer encoders to generate state space representations of mental distress and social rhythm disruption. Such a probabilistic transformer can model temporal state transitions between high and low states of mental distress and social rhythm disruption, and the probabilistic transformer can model changes in the dyadic biomarker representations as indicators of the latent state variables.

As illustrated in FIG. 1, dyadic phenotype signal extractor 152 includes mental distress signal learning module 154 and social rhythm signal learning module 156. In one or more embodiments, mental distress signal learning module 154 module identifies one or more prognostic biomarkers (e.g., predictors) of the level of mental distress (e.g., on a scale of 0 to 100) of a patient at a given time, controlling for the patient's caregiver's effects. In such an embodiment, the mental distress signal learning module 154 also identifies one or more prognostic biomarkers (e.g., predictors) of the level of mental distress of a caregiver at a given time, controlling for the caregiver's patient's effects. The mental distress signal learning module 154 can identify candidate prognostic biomarkers of mental distress, for example, by way of learning a predictive model (such as, e.g., one or more gradient boosted decision trees) and using the model's feature importance ranking mechanism to measure the contribution of individual features (relative to other features in the model) in predicting mental distress.

Additionally, in at least one embodiment, social rhythm signal learning module 156 identifies one or more biomarkers that predict social rhythm disruption (e.g., on a scale of 0 to 100) of a patient while controlling for the patient's caregiver's effects, and similarly for the caregiver's while controlling for the caregiver's patient's effects. The social rhythm signal learning module 156 can identify candidate prognostic biomarkers of social rhythm disruption by, for example, learning a predictive model (such as, e.g., one or more gradient boosted decision trees) and using the model's feature importance ranking mechanism to measure the contribution of individual features in predicting social rhythm disruption.

As also depicted in FIG. 1, extracted signals 158 can include mental distress signals (“distress signals”) and social rhythm signals. In one or more embodiments, mental distress signals can include a time series observation (e.g., related to at least one time-dependent variable) indicating one or more mental distress levels measured on a continuous scale, along with lower and upper bound limits of the 100-a confidence interval (e.g., 95% confidence interval) of the value at each timepoint. In such an embodiment, the mental distress levels can first be measured using a psychological distress questionnaire administered repeatedly across multiple time instances to multiple patient-caregiver dyads. The generated data can then be used to train and/or learn a model that predicts the mental distress level(s) from features (e.g., biomarkers) obtained from patient-caregiver dyads. In an example embodiment, two signals are extracted for each dyad, one signal for the patient and one signal for the caregiver.

Additionally, in at least one embodiment, social rhythm signals can include a time series observation (e.g., related to at least one time-dependent variable) indicating one or more social rhythm levels measured on a continuous scale, along with lower and upper bound limits of the 100-a confidence interval (e.g., 95% confidence interval) of the value at each timepoint. In such an embodiment, the social rhythm levels can first be measured using a social rhythm scale that measures engagement levels in one or more routine activities. Such a scale can be administered repeatedly across multiple time instances to multiple patient-caregiver dyads, and the generated data can then be used to train and/or learn a model that predicts the social rhythm disruption using features (e.g., biomarkers) obtained from patient-caregiver dyads. In an example embodiment, two such signals are extracted for each dyad, one signal for the patient and one signal for the caregiver.

As detailed above, FIG. 1 also depicts avatar-mediated interaction system 160, which generates a composite signal, using at least a portion of extracted signals 158. In one or more embodiments, avatar-mediated interaction system 160 generates a composite signal by means of a weighted average of the patient distress signal(s), the patient social rhythm signal(s), the caregiver distress signal(s), and the caregiver social rhythm signal(s). As used herein, a weighted average composite signal refers to a time series of scores that can be generated using an aggregation formular that considers varying degrees of importance of the distress and social rhythm signals at different time points.

In at least one example embodiment, the avatar-mediated interaction system 160 allows one or more domain experts to preset one or more decision rules based on thresholds of a composite signal. Such threshold-based decision rules trigger events and/or generate recommendations such as, for example, sending requests for avatar-mediated distanced interactions between isolated patients and their caregivers, sending requests for mediation by facility staff, triggering and/or initiating social activities, etc. In one or more embodiments, proposed reactions and/or interventions can be evaluated and rewarded by facility staff (e.g., via at least one reinforcement learning technique). By way of example, based on an individual's state of mental distress and social rhythm disruption (e.g., environmental state), and his or her reactions and/or interventions (e.g., actions), a reward function can be used to map the state-action pairs to a value representing the desirability of the state and response by the individual. Learned experience can then be transferred to patients and/or caregivers having similar characteristics.

Accordingly, as detailed herein, in at least one embodiment, trajectory modeling of extracted dyadic signals can be carried out to identify one or more subgroups of patients and/or caregivers with different mental distress levels, social rhythms as well as events that influence such trajectories and combinations thereof. In such an embodiment, trajectory modeling refers to using methods such as finite mixture models to analyze time series dyadic signals from patient-caregiver dyads to identify one or more subgroups of patient-caregiver dyads that show statistically similar evolutions over time. Additionally or alternatively, one or more embodiments include determining and/or recommending one or more caregivers, among a plurality of caregivers, who can interact with a given patient via avatar-mediated interactions, via robot-mediated interactions, and/or via in-person mediated interactions. In such an embodiment, such recommendations can be made, for example, by way of a classification model and/or a rule-based system.

Also, while one or more embodiments are described herein within the context of patient care facilities (e.g., hospital care settings), additional embodiments can be implemented in connection with contexts such as, for example, caring for remote and/or distant individuals. At least one embodiment can be implemented in connection with a setting such as a hospital room in combination with one or more physical systems such as, for example, a robotic arm which allows the caretaker to carry out physical tasks for the patient (e.g., handing a water bottle or a remote control, picking up headphones which fell to the floor, etc.). One or more embodiments, as detailed herein, can also be implemented to facilitate and/or allow patients and caregivers to carry out interactive social activities (e.g., playing a game of chess, watching a movie together, etc.).

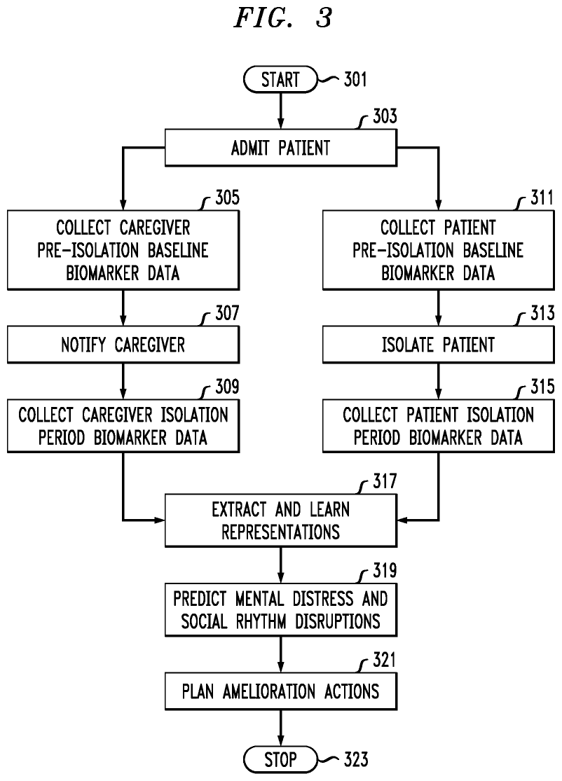

FIG. 3 is a flow diagram illustrating techniques according to an embodiment of the present invention. By way of illustration, the noted techniques start in step 301, and step 303 includes admitting a patient. Subsequent to step 303, step 305 includes collecting caregiver (e.g., a caregiver associated with the admitted patient) pre-isolation baseline biomarker data, and step 311 includes collecting patient pre-isolation baseline biomarker data. In connection with step 311, step 313 includes isolating the patient and step 315 includes collecting patient isolation period biomarker data. Similarly, in connection with step 305, step 307 includes notifying the caregiver and step 309 includes collecting caregiver isolation period biomarker data.

Based at least in part on data collected in steps 309 and 315, step 317 includes extracting and learning one or more representations (from such collected data). Further, step 319 includes predicting (using at least the one or more representations) one or more states of mental distress and/or social rhythm disruptions, and step 321 includes planning one or more amelioration actions (in connection with the predictions). Finally, as depicted in FIG. 3, the techniques stop in step 323.

At least one embodiment may provide a beneficial effect such as, for example, analyzing dyadic data, which enables accounting for interdependence of mental distress and social rhythm disruption between patients and their caregivers. Additionally, one or more embodiments may provide a beneficial effect such as, utilizing artificial intelligence-based methods (e.g., probabilistic transformers) to analyze objectively measured dyadic sequence data, which facilitates and/or enables rapid detection of latent states of mental distress and social rhythm disruptions, and which can be used to predict possible transitions between states.

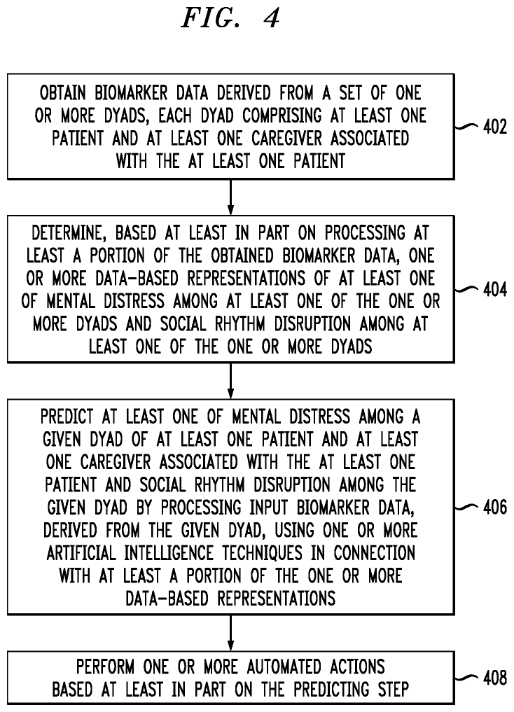

FIG. 4 is a flow diagram illustrating techniques according to an embodiment of the present invention. Step 402 includes obtaining biomarker data derived from a set of one or more dyads, each dyad comprising at least one patient and at least one caregiver associated with the at least one patient. In at least one embodiment, obtaining biomarker data includes obtaining biomarker data from the set of one or more dyads, wherein at least a portion of each dyad comprises at least one isolation-related context.

Step 404 includes determining, based at least in part on processing at least a portion of the obtained biomarker data, one or more data-based representations of at least one of mental distress among at least one of the one or more dyads and social rhythm disruption among at least one of the one or more dyads. In one or more embodiments, determining the one or more data-based representations includes performing phenotypic characterization by processing the at least a portion of the obtained biomarker data using one or more artificial intelligence-based representation learning techniques.

Step 406 includes predicting at least one of mental distress among a given dyad of at least one patient and at least one caregiver associated with the at least one patient and social rhythm disruption among the given dyad by processing input biomarker data, derived from the given dyad, using one or more artificial intelligence techniques in connection with at least a portion of the one or more data-based representations. In at least one embodiment, predicting includes processing input biomarker data, derived from the given dyad, using one or more multivariate time series modeling techniques with one or more probabilistic transformers, in connection with the at least a portion of the one or more data-based representations. Additionally or alternatively, predicting can include processing input biomarker data, derived from the given dyad, using one or more trajectory modeling techniques in connection with the at least a portion of the one or more data-based representations.

Also, in at least one embodiment, predicting includes predicting one or more temporal state transitions associated with at least one of mental distress among the given dyad and social rhythm disruption among the given dyad. Further, in at least one embodiment, processing input biomarker data includes processing input biomarker data derived from the given dyad, and wherein at least a portion of the given dyad comprises at least one isolation-related context.

Step 408 includes performing one or more automated actions based at least in part on the predicting step. In one or more embodiments, performing one or more automated actions includes automatically initiating, based at least in part on the predicting of at least one of mental distress among the given dyad and social rhythm disruption among the given dyad, one or more avatar-mediated interactions within at least a portion of the given dyad, one or more robot-mediated interactions within at least a portion of the given dyad, and/or one or more human-mediated interactions within at least a portion of the given dyad. Also, in at least one embodiment, performing one or more automated actions includes automatically training at least a portion of the one or more artificial intelligence techniques using feedback related to the predicting of at least one of mental distress among the given dyad and social rhythm disruption among the given dyad. Additionally or alternatively, performing one or more automated actions can include generating and outputting, using one or more user interfaces, one or more visualizations pertaining to the predicting of at least one of mental distress among the given dyad and social rhythm disruption among the given dyad.

Also, in one or more embodiments, software implementing the techniques depicted in FIG. 4 can be provided as a service in a cloud environment.

It is to be appreciated that “model,” as used herein, refers to an electronic digitally stored set of executable instructions and data values, associated with one another, which are capable of receiving and responding to a programmatic or other digital call, invocation, or request for resolution based upon specified input values, to yield one or more output values that can serve as the basis of computer-implemented recommendations, output data displays, machine control, etc. Persons of skill in the field find it convenient to express models using mathematical equations, but that form of expression does not confine the models disclosed herein to abstract concepts; instead, each model herein has a practical application in a computer in the form of stored executable instructions and data that implement the model using the computer.

The techniques depicted in FIG. 4 can also, as described herein, include providing a system, wherein the system includes distinct software modules, each of the distinct software modules being embodied on a tangible computer-readable recordable storage medium. All of the modules (or any subset thereof) can be on the same medium, or each can be on a different medium, for example. The modules can include any or all of the components shown in the figures and/or described herein. In an embodiment of the invention, the modules can run, for example, on a hardware processor. The method steps can then be carried out using the distinct software modules of the system, as described above, executing on a hardware processor. Further, a computer program product can include a tangible computer-readable recordable storage medium with code adapted to be executed to carry out at least one method step described herein, including the provision of the system with the distinct software modules.

Additionally, the techniques depicted in FIG. 4 can be implemented via a computer program product that can include computer useable program code that is stored in a computer readable storage medium in a data processing system, and wherein the computer useable program code was downloaded over a network from a remote data processing system. Also, in an embodiment of the invention, the computer program product can include computer useable program code that is stored in a computer readable storage medium in a server data processing system, and wherein the computer useable program code is downloaded over a network to a remote data processing system for use in a computer readable storage medium with the remote system.

An embodiment of the invention or elements thereof can be implemented in the form of an apparatus including a memory and at least one processor that is coupled to the memory and configured to perform exemplary method steps.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 500 contains an example of an environment for the execution of at least some of the computer code 526 involved in performing the inventive methods, such as predicting states from patient-caregiver dyadic biomarker data using artificial intelligence. In addition to code 526, computing environment 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and code 526, as identified above), peripheral device set 514 (including user interface (UI) device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

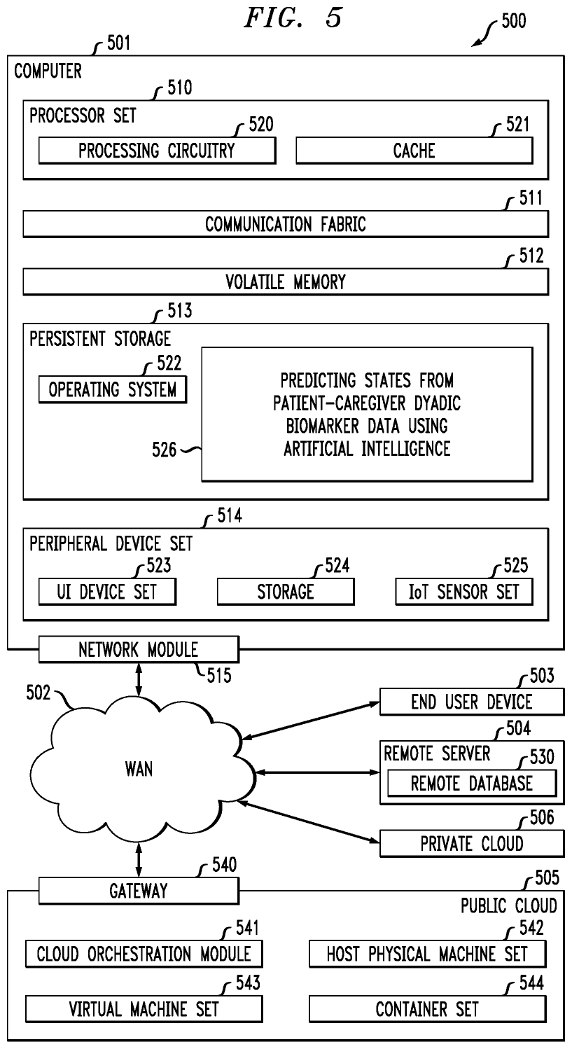

Computer 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of computing environment 500, detailed discussion is focused on a single computer, specifically computer 501, to keep the presentation as simple as possible. Computer 501 may be located in a cloud, even though it is not shown in a cloud in FIG. 5. On the other hand, computer 501 is not required to be in a cloud except to any extent as may be affirmatively indicated.

Processor set 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located “off chip.” In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 521 and the other storage media discussed below. The program instructions, and associated data, are accessed by processor set 510 to control and direct performance of the inventive methods. In computing environment 500, at least some of the instructions for performing the inventive methods may be stored in code 526 in persistent storage 513.

Communication fabric 511 is the signal conduction path that allows the various components of computer 501 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 512 is any type of volatile memory now known or to be developed in the future. Examples include dynamic type RAM or static type RAM. Typically, volatile memory 512 is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

Persistent storage 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a ROM, but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface-type operating systems that employ a kernel. The code included in code 526 typically includes at least some of the computer code involved in performing the inventive methods.

Peripheral device set 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 523 may include components such as a display screen, speaker, microphone, wearable devices (such as goggles and smart watches), keyboard, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 501 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

Network module 515 is the collection of computer software, hardware, and firmware that allows computer 501 to communicate with other computers through WAN 502. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (for example, embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515.

WAN 502 is any wide area network (for example, the internet) capable of communicating computer data over non-local distances by any technology for communicating computer data, now known or to be developed in the future. In some embodiments, the WAN 502 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device 503 is any computer system that is used and controlled by an end user (for example, a customer of an enterprise that operates computer 501), and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments, EUD 503 may be a client device, such as thin client, heavy client, mainframe computer, desktop computer and so on.

Remote server 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530 of remote server 504.

Public cloud 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of VCEs will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of another feature, step, operation, element, component, and/or group thereof.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
obtaining biomarker data derived from a set of one or more dyads, each dyad comprising at least one patient and at least one caregiver associated with the at least one patient;
determining, based at least in part on processing at least a portion of the obtained biomarker data, one or more data-based representations of at least one of mental distress among at least one of the one or more dyads and social rhythm disruption among at least one of the one or more dyads;
predicting at least one of mental distress among a given dyad of at least one patient and at least one caregiver associated with the at least one patient and social rhythm disruption among the given dyad by processing input biomarker data, derived from the given dyad, using one or more artificial intelligence techniques in connection with at least a portion of the one or more data-based representations; and
performing one or more automated actions based at least in part on the predicting step;
wherein the method is carried out by at least one computing device.

2. The computer-implemented method of claim 1, wherein determining the one or more data-based representations comprises performing phenotypic characterization by processing the at least a portion of the obtained biomarker data using one or more artificial intelligence-based representation learning techniques.

3. The computer-implemented method of claim 1, wherein predicting comprises processing input biomarker data, derived from the given dyad, using one or more multivariate time series modeling techniques with one or more probabilistic transformers, in connection with the at least a portion of the one or more data-based representations.

4. The computer-implemented method of claim 1, wherein performing one or more automated actions comprises automatically initiating, based at least in part on the predicting of at least one of mental distress among the given dyad and social rhythm disruption among the given dyad, one or more avatar-mediated interactions within at least a portion of the given dyad.

5. The computer-implemented method of claim 1, wherein performing one or more automated actions comprises automatically initiating, based at least in part on the predicting of at least one of mental distress among the given dyad and social rhythm disruption among the given dyad, one or more robot-mediated interactions within at least a portion of the given dyad.

6. The computer-implemented method of claim 1, wherein performing one or more automated actions comprises automatically initiating, based at least in part on the predicting of at least one of mental distress among the given dyad and social rhythm disruption among the given dyad, one or more human-mediated interactions within at least a portion of the given dyad.

7. The computer-implemented method of claim 1, wherein predicting comprises processing input biomarker data, derived from the given dyad, using one or more trajectory modeling techniques in connection with the at least a portion of the one or more data-based representations.

8. The computer-implemented method of claim 1, wherein predicting comprises predicting one or more temporal state transitions associated with at least one of mental distress among the given dyad and social rhythm disruption among the given dyad.

9. The computer-implemented method of claim 1, wherein performing one or more automated actions comprises automatically training at least a portion of the one or more artificial intelligence techniques using feedback related to the predicting of at least one of mental distress among the given dyad and social rhythm disruption among the given dyad.

10. The computer-implemented method of claim 1, wherein performing one or more automated actions comprises generating and outputting, using one or more user interfaces, one or more visualizations pertaining to the predicting of at least one of mental distress among the given dyad and social rhythm disruption among the given dyad.

11. The computer-implemented method of claim 1, wherein obtaining biomarker data comprises obtaining biomarker data from the set of one or more dyads, wherein at least a portion of each dyad comprises at least one isolation-related context.

12. The computer-implemented method of claim 1, wherein processing input biomarker data comprises processing input biomarker data derived from the given dyad, and wherein at least a portion of the given dyad comprises at least one isolation-related context.

13. The computer-implemented method of claim 1, wherein software implementing the method is provided as a service in a cloud environment.

14. A computer program product comprising a computer readable storage medium having program instructions embodied therewith, the program instructions executable by a computing device to cause the computing device to:
obtain biomarker data derived from a set of one or more dyads, each dyad comprising at least one patient and at least one caregiver associated with the at least one patient;
determine, based at least in part on processing at least a portion of the obtained biomarker data, one or more data-based representations of at least one of mental distress among at least one of the one or more dyads and social rhythm disruption among at least one of the one or more dyads;
predict at least one of mental distress among a given dyad of at least one patient and at least one caregiver associated with the at least one patient and social rhythm disruption among the given dyad by processing input biomarker data, derived from the given dyad, using one or more artificial intelligence techniques in connection with at least a portion of the one or more data-based representations; and
perform one or more automated actions based at least in part on the predicting step.

15. The computer program product of claim 14, wherein determining the one or more data-based representations comprises performing phenotypic characterization by processing the at least a portion of the obtained biomarker data using one or more artificial intelligence-based representation learning techniques.

16. The computer program product of claim 14, wherein predicting comprises processing input biomarker data, derived from the given dyad, using one or more multivariate time series modeling techniques with one or more probabilistic transformers, in connection with the at least a portion of the one or more data-based representations.

17. The computer program product of claim 14, wherein performing one or more automated actions comprises automatically initiating, based at least in part on the predicting of at least one of mental distress among the given dyad and social rhythm disruption among the given dyad, one or more avatar-mediated interactions within at least a portion of the given dyad.

18. A system comprising:
a memory configured to store program instructions; and
a processor operatively coupled to the memory to execute the program instructions to:
obtain biomarker data derived from a set of one or more dyads, each dyad comprising at least one patient and at least one caregiver associated with the at least one patient;
determine, based at least in part on processing at least a portion of the obtained biomarker data, one or more data-based representations of at least one of mental distress among at least one of the one or more dyads and social rhythm disruption among at least one of the one or more dyads;
predict at least one of mental distress among a given dyad of at least one patient and at least one caregiver associated with the at least one patient and social rhythm disruption among the given dyad by processing input biomarker data, derived from the given dyad, using one or more artificial intelligence techniques in connection with at least a portion of the one or more data-based representations; and
perform one or more automated actions based at least in part on the predicting step.

19. The system of claim 18, wherein determining the one or more data-based representations comprises performing phenotypic characterization by processing the at least a portion of the obtained biomarker data using one or more artificial intelligence-based representation learning techniques.

20. The system of claim 18, wherein predicting comprises processing input biomarker data, derived from the given dyad, using one or more multivariate time series modeling techniques with one or more probabilistic transformers, in connection with the at least a portion of the one or more data-based representations.
